(19) United States
(12) Patent Application Publication
(10) Pub. No.: US 2026/0076544 A1
(43) Pub. Date: Mar. 19, 2026

(54) METHOD AND APPARATUS FOR DETERMINING TYPE OF CATARACT

(71) Applicant: SAMSUNG ELECTRONICS CO., LTD., Suwon-si (KR)

(72) Inventors: Vijayanand KUMAR, Noida (IN); Govind GAUR, Noida (IN); Bharat GUPTA, Noida (IN)

(73) Assignee: SAMSUNG ELECTRONICS CO., LTD., Suwon-si (KR)

(21) Appl. No.: 19/402,234

(22) Filed: Nov. 26, 2025

Related U.S. Application Data
(63) Continuation of application No. PCT/KR2023/011829, filed on Aug. 10, 2023.

(30) Foreign Application Priority Data
May 30, 2023 (IN) 202311037223

(51) Int. Cl. A61B 3/00; A61B 3/024

(52) U.S. Cl. CPC A61B 3/0091; A61B 3/0025; A61B 3/0041; A61B 3/024

(57) ABSTRACT

There is provided a method for determining type of cataract by an electronic device. The method includes performing vision focus calibration for a user, determining central and peripheral regions of interest on a display of the electronic device based on fixated vision, performing a primary test for obtaining scores for the central and peripheral regions of interest, selecting at least one vision test mode based on the obtained score, and adapting at least one visual display parameter based on the obtained score and the selected at least one vision test mode. The method also includes determining a type of cataract from the selected at least one vision test mode, determining severity of the determined type of cataract, and providing the determined type of cataract and the determined severity with one or more recommendations to the user.

### CROSS-REFERENCE TO RELATED APPLICATIONS

This application is a continuation of International Application No. PCT/KR2023/011829, filed on Aug. 10, 2023, which is based on and claims priority to Indian Patent Application number 202311037223, filed on May 30, 2023, in the Indian Intellectual Property Office, the disclosures of which are incorporated by reference herein in their entireties.

### BACKGROUND

### 1. Field

The present disclosure relates generally to detecting cataract and more particularly to a method and apparatus for determining a type of the cataract.

### 2. Description of Related Art

A cataract is a common eye disorder that affects millions of people worldwide, and a cataract typically develops slowly over time and may appear in and affect one or both of a person's eyes.

The most common cause of cataracts may be age-related changes in a lens of an eye, although other factors such as genetics, trauma to the eye, and certain medical conditions can also contribute to cataract development. Generally, a cataract may be defined as a progressive clouding of a lens of an eye, which can cause vision impairment and eventually blindness if left untreated. Cataract symptoms include blurred vision, difficulty seeing in bright light, and appearance of halos around lights depending on type of cataract.

A cataract diagnosis may be typically made through a comprehensive eye exam, which may include a visual acuity test, a slit-lamp exam, retinal exam, applanation tonometry, and other diagnostic procedures such as optical coherence tomography (OCT) or ultrasound, any of which are costly, time-consuming and require external assistance. Therefore, in view of such technical difficulties presented with those diagnostic technologies, the optometric and ophthalmic technical fields need an economical and time-saving, non-invasive approach for cataract diagnosis.

Further, treating a cataract may involve surgical removal of the clouded lens and replacement thereof with an artificial lens. Although cataract surgery may be considered to be a safe, highly successful, and widely performed procedure, there are still some risks associated with the surgery, including bleeding, infection, and retinal detachment. Therefore, to avoid the surgery, user should seek regular eye examinations to detect and treat the cataract early, and work closely with an ophthalmologist to ensure best possible outcomes. However, optometric and ophthalmic technical fields are currently limited by the technical difficulties noted above regarding accessibility of such diagnostic devices such that it is not possible for everyone to seek regular eye examinations for and monitoring of cataracts and, hence requires improvement to that technical field, such as by a self-performable method and system for easy, more accessible cataract diagnosis.

In the context of the present optometric technical field, there may be various ways of making a cataract determination or detection.

For example, consider a general vision testing system of devices and computer technology configured for testing the vision of a human subject using a series of eye tests. A test setup procedure of such system may be run to adjust the settings of a display device such that graphic objects displayed on the device conform to a pre-defined appearance. Further, a series of preliminary tests, static tests and dynamic tests may be displayed on the device and the responses of the subject may be recorded. The tests may be run remotely, for example over an internet. No lenses are required to run the tests. However, the optometric technical field has yet to adequately consider possible applications to cataract diagnostics including determining the possible type of cataract or combinational type cataract, its severity, and a way for its validation.

Similarly, consider ophthalmic equipment which may include an interference optical system of an irradiation position changing unit, an analysis unit, and a control unit. The interference optical system may split light from a light source into reference light and measurement light, irradiate the eye with the measurement light, generate interference light between the return light and the reference light, and detect the generated interference light. The irradiation position changing unit may irradiate measurement light to a plurality of positions of the eye to be examined. And the analysis unit may determine the type of cataract based on the intensity distribution of the interference signal corresponding to the detection result of the interference light obtained by the interference optical system at a plurality of positions. And even if a control unit may select a measurement mode of an eye to be examined based on a determination result by the analysis unit the optometric and ophthalmic technical fields are nonetheless silent about an accessible, self-performable visual acuity test for determining the type of cataract and its severity.

Therefore, in light of the foregoing discussion, there exists a need to overcome the aforementioned drawbacks in technical fields associated with the systems and method for accurately and accessibly determining and assessing a type and severity of a cataract.

### DISCLOSURE OF INVENTION

### Solution to Problem

There is provided a method for determining a type of cataract by an electronic device, the method including: performing a vision focus calibration for a user, the vision focus calibration including determining a user vision of the user to be fixated a display of the electronic device based on monitoring the user vision relative to the display and while adjusting an output of the display; determining, based on implementing a further monitoring of the user vision relative to the display and in a state in which it has been determined that the user vision is fixated to the display by the vision focus calibration, central and peripheral regions of interest on the display of the electronic device, the central and peripheral regions of interest being regions of interest of the user vision relative to the display; performing a primary test including: obtaining a score based on scoring the central and peripheral regions of interest determined based on the further monitoring the user vision relative to the display, selecting, from a plurality of vision test modes and based on the obtained score, at least one vision test mode, and adapting, based on the obtained score and the selected at least one vision test mode, at least one visual display parameter relative to the output of the display; determining, based on implementing an even further monitoring of the user vision relative to the display and in a state in which the at least one visual display parameter of the output of the display is adapted based on the obtained score and the selected at least one vision test mode, both the type of cataract of the user and a severity of the determined type of cataract; and controlling the display to provide an indication of the determined type of cataract, the determined severity, and one or more recommendations to the user.

The vision focus calibration may include determining the user vision to be fixated to a center of the display based on any of estimating an eye distance of one or more eyes of the user from the display, monitoring a focus angle from any of the one or more eyes of the user relative to the display, implementing movement tracking of detecting shifts in any of an eye position and an interpupillary distance of the user, and monitoring one or more facial features of the user.

Each of determining the central and peripheral regions of interest, performing the primary test, and implementing the even further monitoring of the user vision may be performed while, by the electronic device monitoring the user vision relative to the display, each of the user vision is determined to be fixated to a center of the display, an eye distance from the display is determined to remain unchanged, and at least one environmental condition is determined to remain unchanged.

The electronic device monitoring the user vision relative to the display while each of the user vision is determined to be fixated to the center of the display, the eye distance from the display is determined to remain unchanged, and the at least one environmental condition is determined to remain unchanged, may include: monitoring and analyzing the user vision, relative to the display for fixation to the center of the display and for the eye distance from the display, by a camera of the electronic device and at least one machine learning model trained by artificial intelligence, and monitoring the at least one environmental condition by at least one sensor in communication with the electronic device.

The display may be rectangular and includes a first side and a second side opposite to the first side along a longitudinal axis of the display, the first side and the second side being ones of right and left sides of the display, and the primary test may represent a sharpness test including: displaying lines and digits on the central and peripheral regions of interest of the display, wherein at least one first line and at least one first digit is output at the first side of the display and relative to monitoring a first eye of the user, and wherein at least one second line and at least one second digit is output at the second side of the display and relative to monitoring a second eye of the user; receiving at least one input from the user in response to the displayed lines and digits while the user vision is determined to be fixated to a center of display; scoring the central and peripheral regions of interest respectively for the first eye and for the second eye; and estimating the type of cataract based on determining whether there is an inequality in scoring the central and peripheral regions of interest for the first eye as compared to for the second eye.

The primary test may be a monocular vision test including controlling the output of the display to display lines and digits in ones of near, mid, and far peripheral areas represented by three circular zones marked on the display by the output of the display.

The selected at least one vision test mode may include at least one of a glare test, a color distortion test, and a night vision test, and the at least one visual display parameter includes at least one of a background color, a line format, a line color, a line thickness, a peripheral display, an external lighting environment, and a display brightness.

The glare test may include, in a glare mode display of the display, controlling the output of the display by changing a background, a color gradient of lines in digits displayed relative to the background and in the central and peripheral regions of interest, and adapting visual display parameters including a glare effect, a brightness, a seven segment line display format, line colors that are a gradient of background colors and are predetermined to be visible to a normal vision, thickness weights of the lines, a size of a peripheral display to be larger moreso than that of a central display, the external lighting environment, and ranges of display brightness.

The color distortion test may include, in a color mode display of the display, changing a background, a color gradient of lines in digits displayed relative to the background and in the central and peripheral regions of interest, and adapting visual display parameters without glare and sharpness effect, a seven segment line display format, line colors that are a gradient of background colors and are predetermined to be visible to a normal vision, thickness weights of the lines, a size of a peripheral display to be larger than that of a central display, an average external lighting condition, and ranges of display brightness.

The night vision may include, in a low light mode display of the display, changing a background, a color gradient of lines in digits displayed relative to the background and in the central and peripheral regions of interest, and adapting visual display parameters without glare and sharpness effect, a seven segment line display format, line colors that are a gradient of background colors and are predetermined to be visible to a normal vision, thickness weights of the lines, a size of a peripheral display to be larger than that of a central display, an average external lighting condition, and ranges of display brightness.

Determining the type of cataract may be further based on obtaining scores, including the score, of the central and peripheral regions of interest based on a plurality of weighing factors, a closeness of a response of the user vision relative to displayed lines and digits on the display, and a confidence level, and the type of cataract may indicate one of a posterior subcapsular cataract (C1), a cortical cataract (C2), a nuclear sclerotic cataract (C3), a combination cataract (C1+C2, C2+C3, and/or C1+C3), and a normal vision.

Performing the primary test may include: determining the type of cataract based on performing the primary test; and iterating the primary test based on determining a difference between the type of cataract as determined based on the primary test as compared to the type of cataract as determined based on implementing the even further monitoring of the user vision relative to the display and in the state in which the at least one visual display parameter of the output of the display is adapted based on the obtained score and the selected at least one vision test mode.

The one or more recommendations may include at least one recommendation indicating a cataract type, an estimated time for surgery, an estimated time to retest for monitoring progression of cataract, a post-surgery test for analyzing improvement in vision, nutrients and food intake, lifestyle and day-to-day activities, and display settings based on any of a gender and a profession of the user.

There is provided an electronic device for determining a type of cataract, the electronic device including: a memory; a display; and a controller coupled to the memory and the display, wherein the controller is configured to: perform a vision focus calibration for a user, the vision focus calibration including determining a user vision of the user to be fixated a display of the electronic device based on monitoring the user vision relative to the display and while adjusting an output of the display; determine, based on implementing a further monitoring of the user vision relative to the display and in a state in which it has been determined that the user vision is fixated to the display by the vision focus calibration, central and peripheral regions of interest on the display of the electronic device, the central and peripheral regions of interest being regions of interest of the user vision relative to the display; perform a primary test including: obtaining a score based on scoring the central and peripheral regions of interest determined based on the further monitoring the user vision relative to the display, selecting, from a plurality of vision test modes and based on the obtained score, at least one vision test mode, and adapting, based on the obtained score and the selected at least one vision test mode, at least one visual display parameter relative to the output of the display; determine, based on implementing an even further monitoring of the user vision relative to the display and in a state in which the at least one visual display parameter of the output of the display is adapted based on the obtained score and the selected at least one vision test mode, both the type of cataract of the user and a severity of the determined type of cataract; and control the display to provide an indication of the determined type of cataract, the determined severity, and one or more recommendations to the user.

The controller may be further configured to implement the vision focus calibration further based on tracking any of: one or more eyes of the user, and facial features of the user.

The controller may be further configured to monitor whether an environmental condition among the user and the display remains unchanged during a process including at least one of: determining the central and peripheral regions of interest, performing the primary test, and implementing the even further monitoring of the user vision.

The primary test may include: monitoring a first eye of the user relative to a first portion of the output of the display, and monitoring a second eye of the user relative to a second portion of the output of the display, wherein the first portion and the second portion are respectively at ones of opposite sides of the display.

The selected at least one vision test mode may include controlling the output of the display to be output in one of a glare mode display of the display, a color mode display of the display, and a low light mode display of the display.

Performing the primary test may include determining that the primary test and the even further monitoring in the selected at least on vision test mode result in different determinations of the type of cataract, and, based thereon, improving an accuracy of determining the type of cataract by reiterating the primary test and re-determining the type of cataract based on reiterating the primary test.

There is provided a non-transitory computer readable storage medium storing instructions. The instructions, when executed by at least one processor of an electronic device, may cause the electronic device to perform a vision focus calibration for a user; determine central and peripheral regions of interest on a display of the electronic device based on a fixated vision of the user; perform a primary test comprising: obtaining a score based on scoring the central and peripheral regions of interest, selecting, from a plurality of vision test modes and based on the obtained score, at least one vision test mode, and adapting, based on the obtained score and the selected at least one vision test mode, at least one visual display parameter; determine the type of cataract of the user and a severity of the determined type of cataract based on the selected at least on vision test mode and the adapted at least one visual display parameter; and providing information about an indication of the determined type of cataract, information about the determined severity, and information about one or more recommendations to the user.

The foregoing summary is illustrative only and is not intended to be in any way limiting. In addition to the illustrative aspects, embodiments, and features described earlier, further aspects, embodiments, and features will become apparent by reference to the drawings and the following detailed description.

### BRIEF DESCRIPTION OF DRAWINGS

The accompanying drawings, which are incorporated herein and constitute a part of this disclosure, illustrate exemplary embodiments, and together with the description, serve to explain the disclosed principles. The same numbers are used throughout the figures to reference like features and components, wherein:

FIG. 1 depicts a flow diagram showing a method for determining type of cataract, in accordance with one or more exemplary embodiments of the present disclosure;

FIG. 2 depicts a block diagram of the system performing method for determining type of cataract, in accordance with one or more exemplary embodiments of the present disclosure;

FIG. 3 depicts a pictorial representation of vision focus calibration, in accordance with one or more exemplary embodiments of the present disclosure;

FIG. 4 depicts a pictorial representation of central and peripheral regions of interest on the display, in accordance with one or more exemplary embodiments of the present disclosure;

FIG. 5 depicts a flow diagram showing a method of performing primary test, in accordance with one or more exemplary embodiments of the present disclosure;

FIG. 6 depicts a pictorial representation of an exemplary embodiment of the primary test, in accordance with one or more exemplary embodiments of the present disclosure;

FIG. 7A depicts a pictorial representation of a glare mode display test, in accordance with one or more exemplary embodiments of the present disclosure;

FIG. 7B depicts a pictorial representation of a color mode display test, in accordance with one or more exemplary embodiments of the present disclosure;

FIG. 7C depicts a pictorial representation of a light mode display test, in accordance with one or more exemplary embodiments of the present disclosure;

FIG. 8 depicts a pictorial representation of an exemplary embodiment of vision test modes performed in peripheral region of interest and score scale, in accordance with one or more exemplary embodiments of the present disclosure;

FIG. 9 depicts a pictorial representation of an exemplary embodiment of severity scale, in accordance with one or more exemplary embodiments of the present disclosure;

FIG. 10 depicts working of a recommendation module, in accordance with one or more exemplary embodiments of the present disclosure;

FIG. 11A depicts a first use case of determining cataract type, in accordance with one or more exemplary embodiments of the present disclosure;

FIG. 11B depicts a second use case involving storage of cataract progression rate, in accordance with one or more exemplary embodiments of the present disclosure;

FIG. 11C depicts a third use case involving determination of post-surgery cataract status, in accordance with one or more exemplary embodiments of the present disclosure; and

FIG. 12 depicts a block diagram of an electronic device in accordance with one or more exemplary embodiments of the present disclosure.

### MODE FOR THE INVENTION

In the following description, numerous specific details are set forth in order to provide a thorough understanding of the present disclosure. It will be apparent, however, to one skilled in the art that these specific details are only exemplary and not intended to be limiting. Additionally, it may be noted that the systems and/or methods are shown in block diagram form only in order to avoid obscuring the present disclosure. It is to be understood that various omissions and substitutions of equivalents may be made as circumstances may suggest or render expedient to cover various applications or implementations without departing from the spirit or the scope of the present disclosure. Further, it is to be understood that the phraseology and terminology employed herein are for the purpose of clarity of the description and should not be regarded as limiting.

Furthermore, in the present description, references to “one embodiment” or “an embodiment” means that a particular feature, structure, or characteristic described in connection with the embodiment is included in at least one embodiment of the present disclosure. The appearance of the phrase “in one embodiment” in various places in the specification is not necessarily referring to the same embodiment, nor are separate or alternative embodiments mutually exclusive of other embodiments. Further, the terms “a” and “an” used herein do not denote a limitation of quantity, but rather denote the presence of at least one of the referenced items. Moreover, various features are described which may be exhibited by some embodiments and not by others. Similarly, various requirements are described, which may be requirements for some embodiments but not for other embodiments.

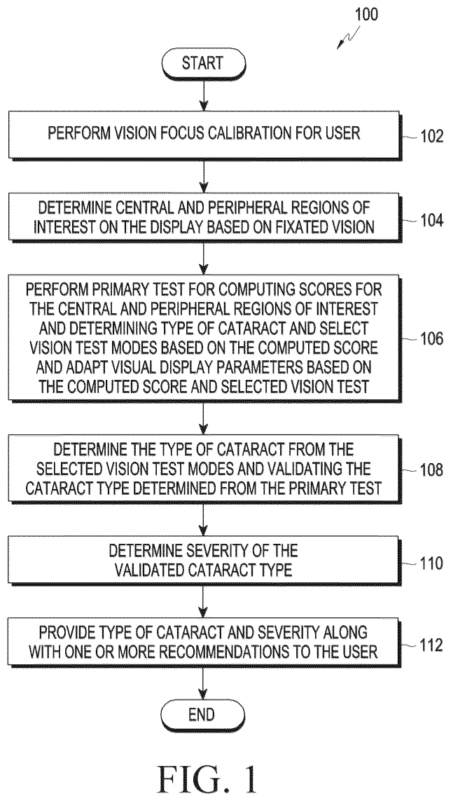

Referring to FIG. 1, a flow diagram showing a method 100 for determining the type of cataract is disclosed. The cataract is a common eye disorder that results in the clouding of a natural lens of an eye, leading to visual impairment. There are several types of cataracts, including a cataract type 1 (C1) or posterior subcapsular cataract that affects outer capsule of the lens and impacts vision such as cloudiness of central vision, absence of glaring, high color distortion or yellow tinted vision, a cataract type 2 (C2) or cortical cataract that affects the outer layer of the lens and impacts vision such as cloudiness of peripheral vision, spoked like significant glaring, and moderate color distortion, a cataract type 3 (C3) or nuclear sclerotic cataract that affects the center of the lens and impacts vision such as equal cloudiness of both visions, minimal glaring, and no color distortion, and a combination cataract type (e.g., any of C1+C2, C2+C3, and C1+C3 for example).

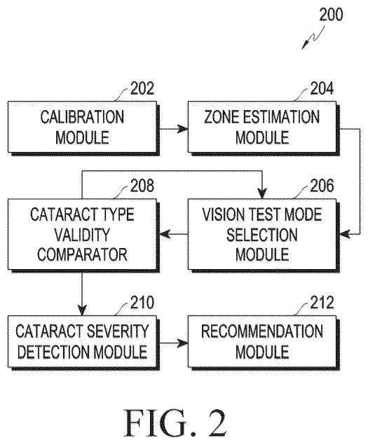

The cataract can develop as a result of a number of factors such as infections during pregnancy, poor diet, due to medical conditions such as diabetes, or as a side effect of certain medications, due to injury to the eye, such as a blow or penetrating injury, etc. The severity of cataracts is typically graded as mild or severe. The severity of cataracts is determined based on the degree of visual impairment, clarity of the lens, and impact on daily activities. Mild cataracts may not cause significant visual impairment and may not require treatment. As the cataract progresses in severity, the visual impairment worsens, and treatment becomes more necessary. Therefore, understanding the type and severity of the cataract is required to determine the appropriate treatment plan, which may include surgery or management of underlying medical conditions. The method of determining the type of cataract may be explained in conjunction with the system disclosed in FIG. 2. In the flow diagram, each block may represent a module, segment, or portion of code, which comprises one or more executable instructions for implementing the specified logical function(s). It should also be noted that in some alternative implementations, the functions noted in the blocks may occur out of the order noted in the drawings. For example, two blocks shown in succession in FIG. 1 may be executed substantially concurrently or the blocks may sometimes be executed in the reverse order, depending upon the functionality involved. Any process descriptions or blocks in flowcharts should be understood as representing modules, segments, or portions of code that include one or more executable instructions for implementing specific logical functions or steps in the process, and alternate implementations are included within the scope of the example embodiments in which functions may be executed out of order from that shown or discussed, including substantially concurrently or in reverse order, depending on the functionality involved. In addition, the process descriptions or blocks in flow charts should be understood as representing decisions made by a hardware structure such as a state machine. The flow diagram starts at step 102 and proceeds to step 112.

At step 102, a vision focus calibration is performed for the user. In one embodiment, the vision focus calibration is performed to fixate user vision at the center of the display of an electronic device. Fixating the user's vision at the center of an electronic device can be achieved through the use of various techniques such as a fixation cross or using point of intersection of horizontal and vertical lines at the center of the display (or UI/UX) of the electronic device. In an exemplary embodiment, the display may be a three-dimensional display enabling provision of a depth or illusion effect, as well as an angular effect, for enhanced visual presentation. In an embodiment, the electronic device includes at least but is not limited to a mobile phone, PDA, computer, laptop, notebook, AR glass, head-mounted display (HMD), and any other device which includes at least but is not limited to a processing module and camera. Further, in the vision focus calibration, one or more vision-related controls are performed using a camera which includes vision fixation of the user on the display, eye distance estimation from the display, focus angle from the eye of the user, movement tracking for detecting shifts in eye(s) position or interpupillary distance, and other facial features.

In an exemplary embodiment, the vision focus calibration is performed in steps including displaying the fixation cross or intersection of horizontal and vertical lines on the display, instructing the user to focus at the center of the intersection, tracking fixation of the eye at the intersection by estimating the view angle, stability of the user (head), eyeball tracking, handling errors in fixation of the eye(s) during focus calibration, primary test, and vision test modes, and continuously correcting and giving instructions to the user to blink, keep focusing on the center, movement error, posture error, etc.

Successively, central and peripheral regions of interest are determined, at step 104, on the display based on fixated vision. In one embodiment, determination of the central and peripheral regions of interest includes determining the circumference of the central vision for the primary test and vision test modes setup and dividing into central and peripheral zones. It should be noted that any error in vision focus calibration, re-initiates determination of the central and peripheral regions of interest.

Successively, a primary test is performed, at step 106. The primary test is performed for computing scores for the central and peripheral regions of interest and to determine the type of cataract. In one embodiment, the primary test is a sharpness test which is performed in steps including displaying lines and digits on the central and peripheral regions of interest of the display. The lines and digits on the left side of the display are for the left eye and the right side is for the right eye. Successively, inputs are received from the user in response to the displayed line and digits while vision is fixated at the center of the display, and thereafter, scores are computed for the central and peripheral regions of interest, and the type of cataract is determined based on inequality relation between the computed scores. In one exemplary embodiment, inputs from the user may be received via a medium such as voice commands or button or touch interfaces on the electronic devices or sensors capable of detecting movements or gestures executed by the user.

In one embodiment, the primary test is a monocular vision test that uses visible lines and digits in near, mid, and far peripheral from three circular zones marked on the display. In another embodiment, the primary test is a binocular vision test that uses visible lines, shapes, and digits anywhere on the display.

Further, the primary test is performed for selecting vision test modes based on the computed score and adapting visual display parameters based on the computed score and selected vision test modes. In one embodiment, the vision test modes include glare test, color distortion test, and night vision test and the visual display parameters include background color, line format, line color, line thickness, peripheral display, external lighting environment, and display brightness. It should be noted that the glare test is performed in glare mode display by changing the background, color gradient of lines in the digits near the background in the central and peripheral regions of interest and adapting the visual display parameters which includes background with glare effect and optimal brightness, seven segment line display format, line colors that are gradient of background colors and are visible to the normal vision, low to high thickness weight of the lines, bigger peripheral display than central display, low external lighting environment, and medium to high range display brightness.

The color mode test is performed in color mode display by changing the background, color gradient of lines in the digits near the background in the central and peripheral regions of interest, and adapting the visual display parameters, which includes a background with no glare and sharpness effect, seven segment line display format, line colors that are gradient of background colors and are visible to the normal vision, low to high thickness weight of the lines, bigger peripheral display than central display, average external lighting condition, and low to medium range display brightness.

The night vision test is performed in low light mode display by changing the background, color gradient of lines in the digits near the background in the central and peripheral regions of interest, and adapting the visual display parameters which includes background with no glare and sharpness effect, seven segment line display format, line colors that are gradient of background colors and are visible to the normal vision, low to high thickness weight of the lines, bigger peripheral display than central display, average external lighting condition, and low to medium range display brightness.

Successively, the type of cataract is determined from the selected vision test modes, and the cataract type determined from the primary test is validated, at step 108. In one embodiment, the type of cataract is determined by computing score for the central and/or peripheral region of interest. The score for the central and peripheral regions of interest is computed based on a plurality of weighing factors, closeness of response for the displayed line and digits, and confidence level, and the type of cataract includes posterior subcapsular cataract (C1), cortical cataract (C2), nuclear sclerotic cataract (C3), combination cataract (C1+C2, C2+C3, and/or C1+C3), and normal vision.

In case the cataract type determined from the vision test modes is not the same as that determined from the primary test, the primary test is iterated until the cataract type determined from the selected vision test modes is consistent with the cataract type determined from the primary test. As such, deficiencies in the computer technology itself, such as the accuracy of the technologies initial and/or intermediate determinations, may be alleviated by improving accuracy through such iterations and determination of consistency.

Successively, severity of the validated cataract type is determined, at step 110, and the type of cataract, severity along with one or more recommendations are provided to the user, at step 112. In one embodiment, the one or more recommendations include recommendations such as information related to the cataract type, estimated time for surgery, estimated time to re-test for monitoring the progression of the cataract, post-surgery test for analyzing improvement in vision, required nutrients and food intake, lifestyle, and day-to-day activities, and display settings as per gender and profession of the user. And as such, not only are the optometric and ophthalmic technical fields improved by such features representing improved practical accessibility over the complicated devices and protocols described in the background herein, but even that accessible form is improved by alleviated deficiencies in the computer's initial and intermediate detection capabilities by features such as at step 106 above.

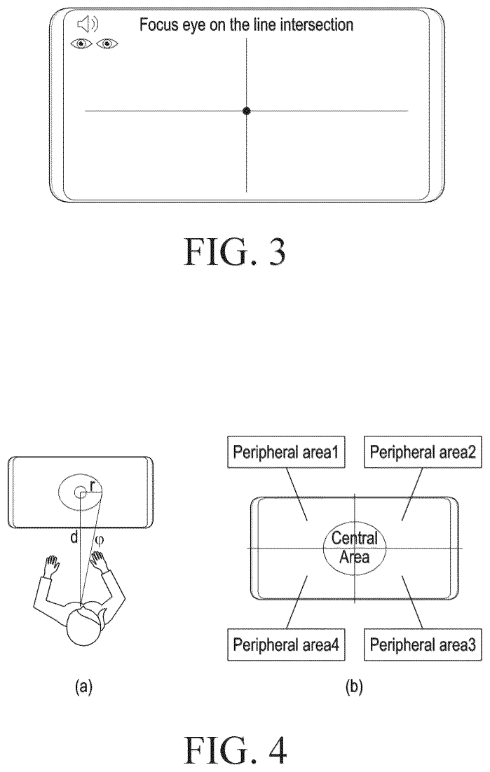

Referring to FIG. 2, a block diagram for determining type of the cataract is disclosed, in accordance with one or more exemplary embodiments of the present disclosure. The system 200 comprises a calibration module 202 for performing vision focus calibration for the user. The vision focus calibration is performed to fixate user vision at the center of the display of the electronic device, which is explained in detail in FIG. 3. The features represented by any one or more of the blocks shown in FIG. 2 may be any of individually and collectively implemented by one or more hardware processors.

Referring to FIG. 3, the pictorial representation of vision focus calibration is depicted. In order to perform the vision focus calibration, the calibration module 202 provides a point of focus at the center of the display which is the point of intersection of horizontal and vertical lines for the user to fixate the vision. In an exemplary embodiment, the calibration module 202 activates the audio source to provide to do instructions to the user to fixate the vision.

The calibration module 202 is further configured for checking ideal testing conditions. It should be noted that the calibration module 202 provides one or more instructions to the user to fixate the vision at the center of the display, maintains constant eye distance from the display, and constant environmental conditions like lighting, glare, etc. In case of any change in one of the user's vision, eye distance from the display, and changes in the environmental conditions, notification of change is provided to the user, and an action is performed by the calibration module 202. The action may include restarting the method 200 for determining the type of cataract or re-conducting the specific steps in which the change is reflected. The change in the user's vision and eye distance from the display is detected using artificial intelligence methodology involving the camera of the electronic device and changes in the environmental conditions are detected using sensors configured with the electronic device.

The calibration module 202 is further configured for determining the level of difficulty the user is facing in fixating the vision at the center. In case of difficulty level is high, the circle at the point of intersection is shifted to either the left or right direction, and the user is instructed to focus on the first visible circle. In another case, where the level of difficulty is low the user is instructed to focus on the intersection. The calibration module 202 is further configured for performing one or more vision-related controls using a camera which includes vision fixation of the user on the display, eye distance estimation from the display, focus angle from the eye of the user, movement tracking for detecting shifts in eye position or interpupillary distance, and other facial features to ensure vision focus calibration.

The system 200 further comprises a zone estimation module 204. The zone estimation module 204 is configured to determine central and peripheral regions of interest on the display based on the fixated vision, which is explained in detail in FIG. 4. The central and peripheral regions of interest are determined to test the user ability to respond to a visual stimuli in the center as well as in the peripheral region.

Referring to FIG. 4, a pictorial representation of central and peripheral regions of interest on the display is depicted. In order to determine the central and peripheral regions of interest on the display, the zone estimation module 204 determines the radius of the central region of interest for a predefined eye distance from the display and predefined tangential angle using a formula given below:

$$tan φ = \frac{r}{d}$$

wherein, φ is a tangential angle, r is the radius of the central region of interest, and d is the eye distance from the display. In an exemplary embodiment, where the predefined eye distance from the display is 25 cm and the predefined tangential angle is 5 degrees, the radius of the central region of interest may be calculated as r=tan 5×25=2.18.

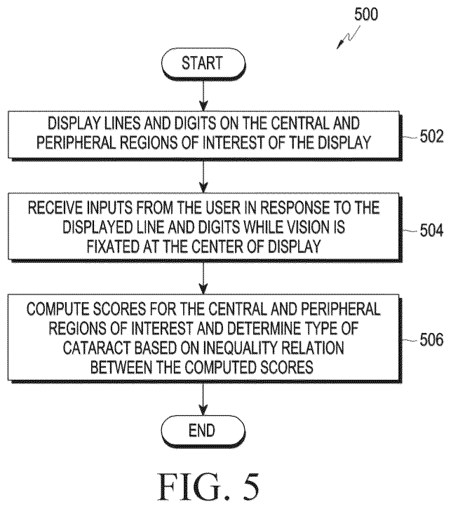

The system 200 further comprises a vision test mode selection module 206. The vision test mode selection module 206 is configured to perform the primary test for computing scores for the central and peripheral regions of interest and determine the type of cataract and select vision test modes based on the computed score and adapt visual display parameters based on the computed score and selected vision test modes, which is explained in detail in FIG. 5. The vision test mode selection module 206 performs the functions in two phases. In the first phase, the primary test is performed for computing scores for the central and peripheral regions of interest and determines the type of cataract. In the second phase, vision test modes are selected based on the computed score from the first phase, and visual display parameters are adapted based on the computed score from the first phase and selected vision test modes. In one embodiment, the primary test is a sharpness test. The sharpness test helps to determine a user's ability to see objects at a distance clearly and is a key component of a comprehensive eye exam. The sharpness test can be affected by a variety of factors, including age, refractive errors, eye diseases, and other medical conditions. By computing the score, cataract can be diagnosed.

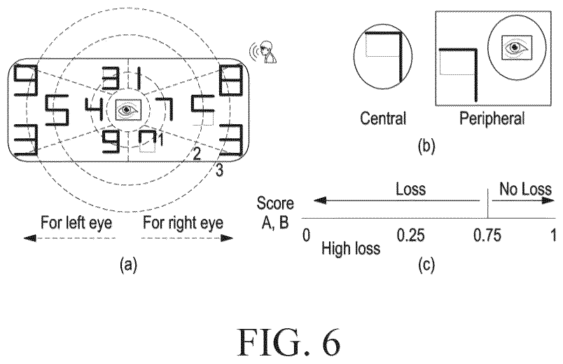

Referring to FIG. 5, a flow diagram showing a method of performing the primary test is depicted. At first, lines and digits are displayed, at step 502 on the central and peripheral regions of interest of the display. In one embodiment, the lines and digits displayed on the left side of the display are for the left eye and displayed on the right side of the display for the right eye, as depicted in the “A” part of FIG. 6. In one exemplary embodiment, the user may be asked to count displayed lines and digits from a specific distance, usually 25 cm. The ratio of height to radius for line is 1.5:1 and the ratio of breadth to radius for the digit is 1:1.

In another embodiment, the user may be asked to identify letters, numbers, or shapes of progressively smaller sizes on an eye chart from a specific distance, usually 25 cm. The standard chart used for this test is the Snellen chart, which has several lines of letters decreasing in size from top to bottom. The top line is usually the largest and represents 20/200 vision, while the bottom line is the smallest and represents 20/10 vision

Successively, inputs are received, at step 504, from the user in response to the displayed line and digits while vision is fixated at the center of display. Thereafter, scores are computed for the central and peripheral regions of interest, and the type of cataract is determined, at step 506. In one embodiment, the score is computed using the formula given below:

$$Score = w [ 0 ] * closeness to the answer + w [ 1 ] * confidence level$$

wherein, w [0] and w [1] are weighing factors of predefined values.

Closeness to the answer may be computed using the formula:

$$\frac{overlapping lines in predicted digit}{total lines in display} \\ or \\ \frac{\frac{1}{1 + abs ( predicted lines - actual lines )} + \frac{overlapping lines in predicted digit}{total lines displayed actual digit}}{2}$$

wherein, abs provide an absolute value; and Confidence level=1−(reply time for line or digit query/max time), if closeness to the answer>threshold value, else confidence level=0, wherein max time is predefined time in seconds.

In an exemplary embodiment, lines and digits are displayed on the central and peripheral regions of interest of the display, as depicted in the “B” part of FIG. 6. The lines and digits displayed on the left side of the display are for testing the central region of interest and displayed on the right side of the display are for testing the peripheral region of interest. The vision test mode selection module (206) on receiving the response for the displayed lines and digits computes the scores using the weighing factors w [0]=0.8 and w [1]=0.20, predefined time=10 sec, and threshold value=0.4 in the above-disclosed formula. The score computed for the central region of interest (A_score_{central region}) is 0.44 and the peripheral region of interest (B_score_{peripheral region}) is 0.98, where A and B are variables used to represent score for the central and peripheral region of interest respectively.

After computing the scores, the type of cataract is determined based on inequality relation between the computed scores using the primary score scale depicted in the “C” part of FIG. 6.

In an exemplary embodiment, the type of cataract is determined from the inequality relation between the computed scores as disclosed in Table 1 given below:

TABLE 1

| Type of | Inequality | |
|---|---|---|
| Cataract | relation | Meaning |
| C1 | Central Loss: | A < 0.75 and B >= 0.75 |
| | B >> A, B≈1 | or nearly 1 |
| C2 | Peripheral loss: | B > 0.75 and A >= 0.75 |
| | A >> B, A≈1 | or nearly 1 |
| C3 | Uniform Loss: | A and B < 0.75 and A |
| | A≈B | and B are nearly close |
| C1 | Unequal loss: | A and B < 0.75 but A is |
| with C3 | 0 < A << B | very less than B |
| C2 | Unequal loss: | A and B < 0.75 but B is |
| with C3 | 0 < B << A | very less than A |
| Normal | No Loss: | A and B > 0.75 and both |
| | A≈1, B≈1 | are nearly close |

Table 1 shows types of cataracts for different inequality relations between the computed scores.

As shown in Table 1, the cataract type is C1 only when there is a central loss i.e. B>>A, B≈1, which means A<0.75 and B>=0.75, or nearly 1.

The cataract type is C2 only when there is a peripheral loss i.e. A>>B, which means A≈1, B>0.75, and A>=0.75, or nearly 1.

The cataract type is C3, only when there is uniform loss i.e. A≈B, which means A and B<0.75 and A and B are nearly close.

The cataract type is C1 with C3, only when there is an unequal loss i.e. 0<A<<B which means A and B are <0.75 but A is very less than B.

The cataract type is C2 with C3, only when there is an unequal loss i.e. 0<B<<A which means A and B are <0.75 but B is very less than A.

There is no cataract or normal vision, only when there is no loss i.e. A≈1, B≈1 which means A and B are >0.75 and both are nearly close.

Normal vision refers to the ability to see clearly and sharply at a standard distance without the need for corrective measures like glasses or contact lenses. Typically, normal vision is rated as 20/20 on an eye chart, which means that the user is able to see digits and lines of the digits from a distance of 25 cm.

It should be noted that normal vision can vary depending on the individual's age, health, and environment.

From the above table, the cataract type for the computed score for the central region of interest (A_score_{central region}) and peripheral region of interest (B_score_{peripheral region}) is determined as C1. i.e.

Type of Cataract=C1, For A_score_{central region}=0.44, and B_score_{peripheral region} 0.98=Zₛₕₐᵣₚₙₑₛₛ

Further, Table 2 discloses the determination of vision loss from the primary test on central and peripheral regions of interest as shown below:

TABLE 2

| Type of | Score (Central | Score (Peripheral | |
|---|---|---|---|
| Cataract | Region of Interest) | Region of Interest) | Vision Loss |
| C1 | Loss | No loss | Strictly loss in |
| | | | central vision |
| C2 | No loss | loss | Strictly loss in |
| | | | peripheral vision |
| C3 | Uniform Loss | | Uniform loss in |
| | | | both the Vision |
| C1 | Major loss: | Minor loss: | Finite Major Loss |
| with C3 | | | in Central Vision |
| C2 | Minor loss: | Major loss: | Finite Major Loss |
| with C3 | | | in Peripheral Vision |

| | | |
|---|---|---|
| Normal | Uniform Loss | No loss in either |
| | | of the Vision |

Table 2 shows determination of vision loss from the primary test on central and peripheral regions of interest.

Table 2 discloses loss/no loss in central and/or peripheral vision for different types of cataracts with respect to the score in the central and peripheral regions of interest.

The vision test mode selection module 206 is further configured for selecting the vision test mode based on the computed score. In one embodiment, the vision test modes include the glare test, color distortion test, and night vision test.

The glare test is performed in glare mode display by changing the background, color gradient of lines in the digits near the background in the central and peripheral regions of interest, the color mode test is performed in color mode display by changing the background, color gradient of lines in the digits near the background in the central and peripheral regions of interest, and the night vision test is performed in low light mode display by changing background, color gradient of lines in the digits near the background in the central and peripheral regions of interest.

The vision test mode selection module 206 is further configured for adapting visual display parameters based on the computed score and selected vision test modes.

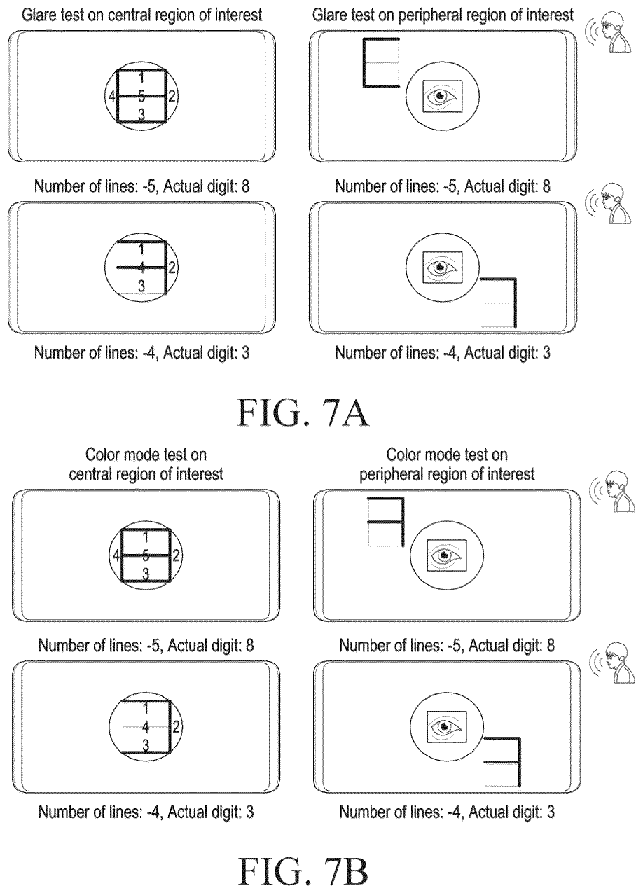

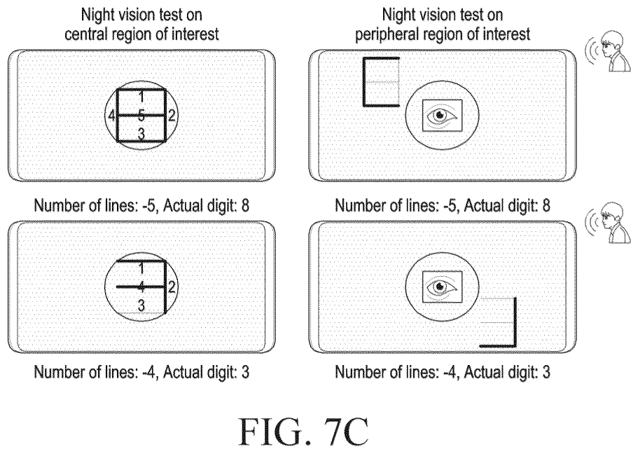

For the selected glare test, the visual display parameters adapted include a background with glare effect and optimal brightness, seven segment line display format, line colors that are a gradient of background colors and are visible to the normal vision, low to high thickness weight of the lines, bigger peripheral display than central display, low external lighting environment, and medium to high range display brightness, as depicted in FIG. 7A.

For the selected color mode test, the visual display parameters adapted include a background with no glare and sharpness effect, seven segment line display format, line colors that are gradient of background colors and are visible to the normal vision, low to high thickness weight of the lines, bigger peripheral display than central display, average external lighting condition, and low to medium range display brightness, as depicted in FIG. 7B.

For the night vision test, the visual display parameters adapted include a background with no glare and sharpness effect, seven segment line display format, line colors that are a gradient of background colors and are visible to the normal vision, low to high thickness weight of the lines, bigger peripheral display than central display, average external lighting condition, and low to medium range display brightness, as depicted in FIG. 7C.

In an exemplary embodiment, the vision test mode selection module 206selects the glare test, the color mode test, and the night vision test only for the peripheral region of interest for the determined cataract type C1.

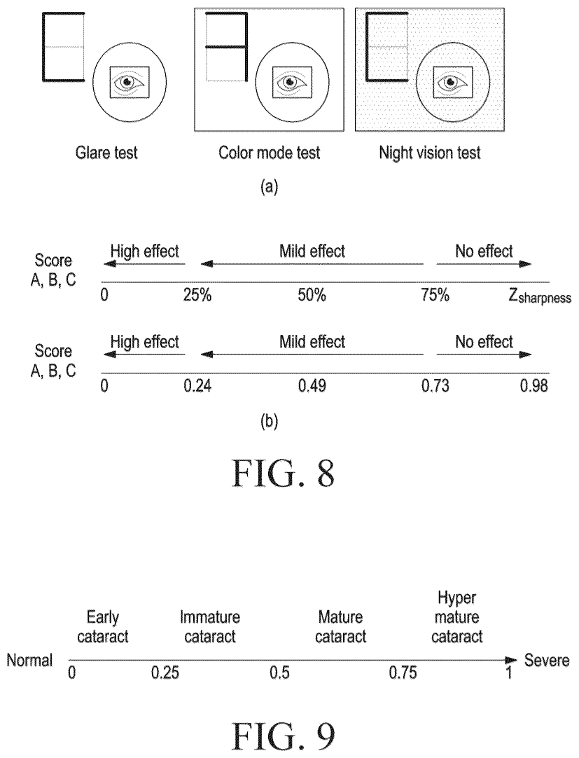

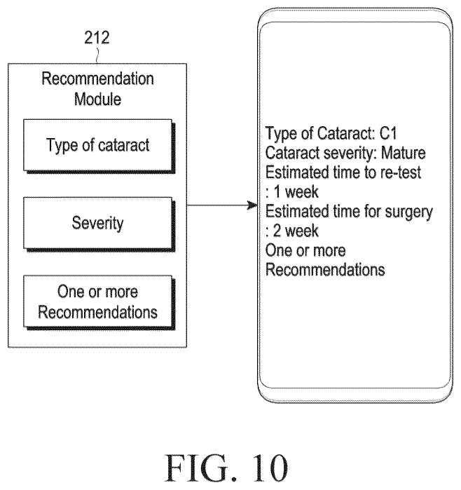

The system 200 further comprises a cataract type validity comparator 208. The cataract type validity comparator 208 is configured for computing score for the central and/or peripheral regions of interest for the selected vision test modes. In an exemplary embodiment, the cataract type validity comparator 208 computes scores for the peripheral region of interest for the glare test, the color mode test, and the night vision test, as explained in FIG. 8.

Referring to FIG. 8, the pictorial representation of an exemplary embodiment of the vision test modes performed in the peripheral region of interest is depicted.

The cataract type validity comparator 208 computes score for the depicted embodiment using the formula based on a plurality of weighing factors, closeness of response for the displayed line and digits, and confidence level as disclosed above and provide A_score_{peripheral region}=0.96, B_score_{peripheral region}=0.20, and C_score_{peripheral region}=0.55, where A, B, and C are scores for glare test, color mode test, and night vision test respectively.

The cataract type validity comparator 208 is further configured for determining the type of cataract.

In one embodiment, the type of cataract is determined based on inequality relation between the computed scores using the score scale depicted in the “B” part of FIG. 8. The type of cataract determined from the inequality relation between the computed scores is disclosed in table 3 as given below:

TABLE 3

| Type of Cataract | Inequality relation |
|---|---|
| C1 | A≈Zₛₕₐᵣₚₙₑₛₛ, B << C, C << Zₛₕₐᵣₚₙₑₛₛ |
| C1 | A≈Zₛₕₐᵣₚₙₑₛₛ, B≈C, B << Zₛₕₐᵣₚₙₑₛₛ, |
| with C3 | C << Zₛₕₐᵣₚₙₑₛₛ |
| Normal | A≈B≈C≈Zₛₕₐᵣₚₙₑₛₛ |

Table 3 shows inequality relation between the computed scores and respected type of cataract.

As shown in Table 3, the cataract type is C1 only when there is A≈Zₛₕₐᵣₚₙₑₛₛ, B<<C, C<<Zₛₕₐᵣₚₙₑₛₛ, the cataract type is C1 with C3 only when there is A≈Zₛₕₐᵣₚₙₑₛₛ, B≈C, B<<Zₛₕₐᵣₚₙₑₛₛ, C<<Zₛₕₐᵣₚₙₑₛₛ, and the cataract type is normal only when there is A≈B≈C≈Zₛₕₐᵣₚₙₑₛₛ.

Further, Table 4 discloses the vision test modes' effect on central and peripheral regions of interest as shown below

TABLE 4

| Type of | | Glare | Color | Night | Inequality |
|---|---|---|---|---|---|
| Cataract | Test | Test | Test | VisionTest | relation |
| C1 | Peripheral | No | High | Mild | A≈Zₛₕₐᵣₚₙₑₛₛ, B << C, |
| | | Effect | Effect | Effect | C << Zₛₕₐᵣₚₙₑₛₛ |
| C2 | Central Region | High | Mild | No | C≈Zₛₕₐᵣₚₙₑₛₛ, A << B, |
| | of Interest | Effect | Effect | Effect | B << Zₛₕₐᵣₚₙₑₛₛ |
| C3 | Peripheral and | No | No | High | A≈B≈Zₛₕₐᵣₚₙₑₛₛ, |
| | Central Regions | Effect | Effect | Effect | C << Zₛₕₐᵣₚₙₑₛₛ |
| C1 | of Interest | No | Mild- | Mild- | A≈Zₛₕₐᵣₚₙₑₛₛ, B≈C, |
| with C3 | | Effect | High | High | B << Zₛₕₐᵣₚₙₑₛₛ, |
| | | | Effect | Effect | C << Zₛₕₐᵣₚₙₑₛₛ |
| C2 | | High | Mild | High | A≈C, B > A, B > C, |
| with C3 | | Effect | Effect | Effect | B << Zₛₕₐᵣₚₙₑₛₛ |
| Normal | | No | No | No | A≈B≈C≈ Zₛₕₐᵣₚₙₑₛₛ |
| | | Effect | Effect | Effect | |

Table 4 shows effect of the vision test modes on the central and peripheral regions of interest.

Table 4 discloses the effects of different vision test modes on the peripheral and central regions of interest and respective cataract type based on inequality relation. For the computed scores i.e. A_score_{peripheral region}=0.96, B_score_{peripheral region}=0.20, and C_score_{peripheral region}=0.55, the type of the cataract is determined as C1 by utilizing the inequality relation disclosed in Table 3 and Table 4.

The cataract type validity comparator 208 is further configured for validating the cataract type determined from the primary test. It should be noted that the cataract type is validated only if the cataract type determined from the vision test modes is the same as that determined from the primary test. In a case where the cataract type determined from the vision test modes is not the same as that determined from the primary test, the primary test is iterated until the cataract type determined from the vision test modes is the same as that determined from the primary test.

Further, it should be noted that the determination of the central and peripheral regions of interest, the primary test, and vision test modes are performed only when the user's vision is fixated at the center of the display, eye distance from the display remains unchanged, and environmental conditions such as lighting conditions remain unchanged. In case of any change, the method 200 for determining the type of cataract or the specific step in which the change is reflected is re-conducted.

The system 200 further comprises a cataract severity detection module 210. The cataract severity detection module 210 is configured for determining severity of the validated cataract type. In one embodiment, the cataract severity detection module 210 computes severity using the formula given below:

$$Cataract Severity {=}^{\sum \frac{1 - Affected Test Mode Scores}{Total Affected Test Mode}}$$

After computing the severity, the severity score is classified in category as early cataract, immature cataract, mature cataract, and hyper mature cataract using the severity scale depicted in FIG. 9. In an exemplary embodiment, the cataract severity for the affected test mode scores which include score A_score_{central region}=0.44 from the primary test and B_score_{peripheral region}=0.20, and C_score_{peripheral region}=0.55 from the vision test modes and three affected test modes is:

$$Cataract severity {=}^{\frac{( 1 - 0.44 ) + ( 1 - 0.2 ) + ( - 0.55 )}{3}} = 0.6$$

Using the severity scale, the severity of cataract type C1 is determined as mature.

The system 200 further comprises a recommendation module 212. The recommendation module 212 is configured for providing the type of cataract and severity along with one or more recommendations to the user in the display of the electronic device. In one embodiment, the one or more recommendations include recommendations such as information related to cataract type, estimated time for surgery, estimated time to re-test for monitoring the progression of the cataract, post-surgery test for analyzing improvement in vision, required nutrients and food intake, lifestyle and day-to-day activities, and display settings as per gender and profession of the user. In one embodiment, the recommendation module 212 retrieves the one or more recommendations from one or more databases based on the validated type of cataract and severity of the cataract. In an exemplary embodiment, the one or more databases include type of cataract, respective recommendations based on the severity of the cataract as disclosed in Table 5 below:

TABLE 5

| | | | | | Hyper |
|---|---|---|---|---|---|
| Type of | Recom- | Early | Immature | Mature | mature |
| Cataract | mendations | Cataract | Cataract | Cataract | Cataract |
| C1 | Re-testing | 1 month | 3 weeks | 1 week | Immediate |
| | due time | | | | surgery |
| | Surgery | 6 months | 2 months | 2 weeks | |
| | due by | | | | |
| C2 | Re-testing | 3 weeks | 2 weeks | Immediate | Immediate |
| | due time | | | surgery | surgery |
| | Surgery | 3 months | 1 month | | |
| | due by | | | | |
| C3 | Re-testing | 3 months | 1 month | 2 weeks | Immediate |
| | due time | | | | surgery |
| | Surgery | 1 year | 6 months | 1 month | |
| | due by | | | | |

| C1 | Re-testing | Depends on dominance of cataract | | Immediate |
|---|---|---|---|---|
| with C3 | due time | typeC1 >> C3: follow C1 | | surgery |
| | Surgery | ruleC3 >> C1: follow C3 rule | | |
| | due by | | | |
| C2 | Re-testing | Depends on dominance | Immediate | Immediate |
| with C3 | due time | of cataract typeC2 >> | surgery | surgery |
| | Surgery | C3: follow C2 | | |
| | due by | ruleC3 >> C2: | | |
| | | follow C3 rule | | |

Table 5 shows recommendations based on the validated type of cataract and severity of the cataract.

As disclosed in Table 5, for the type of cataract C1, the recommendation is retrieved, which includes performing re-testing of the cataract after one month in case of an early cataract, performing re-testing of the cataract after three weeks in case of an immature cataract, performing re-testing of the cataract after one week in case of a mature cataract, and performing immediate surgery in case of a hyper mature cataract. Similarly, for cataract types C2, C3, C1 with C3, and C2 with C3 different recommendations are retrieved from the one or more databases. The “with” of C1 with C3 and C2 with C3 may be considered equivalent to the “+” used at other descriptions herein.

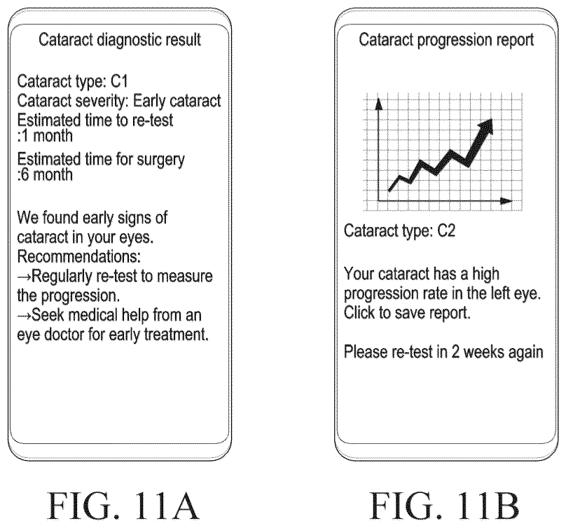

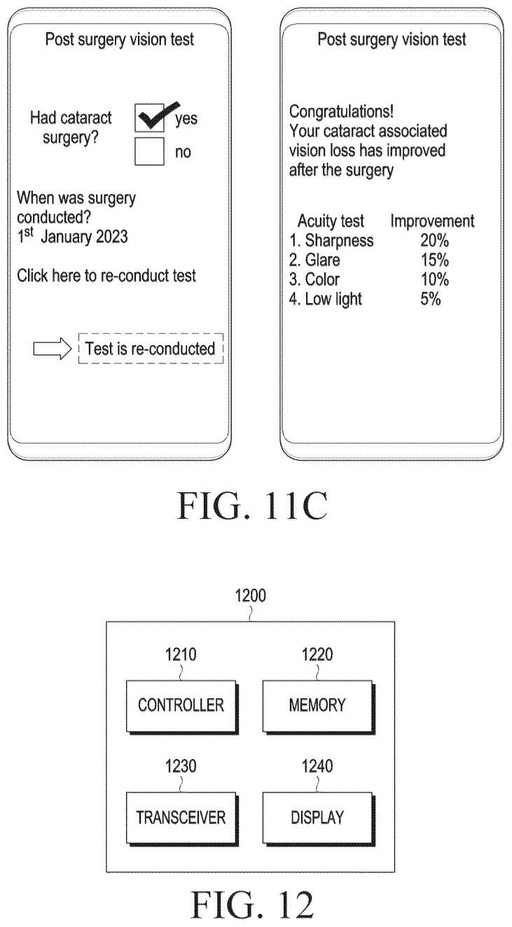

Referring to FIG. 11A, a first use case of determining cataract type is depicted, in accordance with one or more exemplary embodiments of the present disclosure. As depicted, the user is provided with recommendations on the mobile device pertaining to the type and severity of the cataract, estimated timing for a re-test using the present system, estimated timing for surgery, and additional recommendations such as performing re-testing regularly to monitor the progression of the cataract and seek medical attention from an eye specialist for early treatment.

Referring to FIG. 11B, a second use case involving storage of cataract progression rate is depicted, in accordance with one or more exemplary embodiments of the present disclosure. As depicted, the present system maintains a timely record of the severity of the cataract to estimate speed of its progression which allows the user to promptly seek the advice of an eye specialist if the cataract shows signs of spreading really fast. Additionally, it provides criticality of situation on timely manner, and validate slow progression against good eating, habits, medications and other preventive and controlling measures

Referring to FIG. 11C, a third use case involving determination of post-surgery cataract status is depicted, in accordance with one or more exemplary embodiments of the present disclosure. As depicted, the present invention collects data from the user regarding eye surgery and provides a percentage value representing improvement in parameters such as sharpness, glare, color, and low light vision, post-surgery, by carrying out the test using the present system.

Additionally, the system may be used to provide one or more recommendations to the user to adjust the display settings on their mobile device, as well as lifestyle and daily activities, and suggest nutrient and food intake based on the specific type of cataract. Also, if connected to an IOT system, the system can adjust room lights-color, brightness, intensity, and/or alike, which further improves the accessibility and technical fields described herein.

FIG. 12 depicts a block diagram of an electronic device in accordance with one or more exemplary embodiments of the present disclosure.

The electronic device 1200 may perform the methods for detecting the type of cataract according to the embodiments described in the present disclosure. The system 200 may be implemented in the electronic device 1200.

The electronic device 1200 may comprise a controller 1210, a memory 1220, a transceiver 1230, and a display 1240.

The controller 1210 may be implemented through at least one processor. The controller 1210 may control operations of other elements of the electronic device 1200. The controller 1210 may control overall operations of the electronic device 1200. The operations of the electronic device 1200 may be understood as being executed substantially by the controller 1210.

The memory 1220 may store temporary data and/or permanent data for use of the controller 1220. The memory 1220 may store instructions. When the instructions are executed by the controller 1220, the instructions may cause the electronic device 1200 or the controller 1220 to perform operations described in the present disclosure.

The display 1240 may present visual information. The display 1240 may incorporate a touch sensing panel for detecting touch inputs.

It has thus been seen that the system and method for determining type of cataract according to the present invention achieve the purposes highlighted earlier. Such a system and method can in any case undergo numerous modifications and variants, all of which are covered by the same innovative concept, moreover, all of the details can be replaced by technically equivalent elements. The scope of protection of the invention is therefore defined by the attached claims.

What is claimed is:

1. A method for determining a type of cataract by an electronic device, the method comprising:
performing a vision focus calibration for a user;
determining central and peripheral regions of interest on a display based on a fixated vision of the user;
performing a primary test comprising:
obtaining a score based on scoring the central and peripheral regions of interest,
selecting, from a plurality of vision test modes and based on the obtained score, at least one vision test mode, and
adapting, based on the obtained score and the selected at least one vision test mode, at least one visual display parameter;
determining the type of cataract of the user and a severity of the determined type of cataract based on the selected at least on vision test mode and the adapted at least one visual display parameter; and
providing information about the determined type of cataract, information about the determined severity, and information about one or more recommendations to the user.

2. The method of claim 1, wherein performing the vision focus calibration comprises determining a vision of the user to be fixated to a center of the display based on any of estimating an eye distance of one or more eyes of the user from the display, monitoring a focus angle from any of the one or more eyes of the user relative to the display, implementing movement tracking of detecting shifts in any of an eye position and an interpupillary distance of the user, and monitoring one or more facial features of the user.

3. The method of claim 1, wherein each of determining the central and peripheral regions of interest, performing the primary test, and implementing the even further monitoring of the user vision is performed while, by the electronic device monitoring the user vision relative to the display, each of the user vision is determined to be fixated to a center of the display, an eye distance from the display is determined to remain unchanged, and at least one environmental condition is determined to remain unchanged.

4. The method of claim 3, wherein the electronic device monitoring the user vision relative to the display while each of the user vision is determined to be fixated to the center of the display, the eye distance from the display is determined to remain unchanged, and the at least one environmental condition is determined to remain unchanged, comprises:
monitoring and analyzing the user vision, relative to the display for fixation to the center of the display and for the eye distance from the display, by a camera of the electronic device and at least one machine learning model trained by artificial intelligence, and
monitoring the at least one environmental condition by at least one sensor in communication with the electronic device.

5. The method of claim 1,
wherein the display comprises a first side and a second side opposite to the first side,
wherein the primary test represents a sharpness test comprising:
displaying lines and digits on the central and peripheral regions of interest of the display, wherein at least one first line and at least one first digit is output at the first side of the display and relative to monitoring a first eye of the user, and wherein at least one second line and at least one second digit is output at the second side of the display and relative to monitoring a second eye of the user;
receiving at least one input from the user in response to the displayed lines and digits while the user vision is determined to be fixated to a center of display;
scoring the central and peripheral regions of interest respectively for the first eye and for the second eye; and
estimating the type of cataract based on determining whether there is an inequality in scoring the central and peripheral regions of interest for the first eye as compared to for the second eye.

6. The method of claim 1, wherein the primary test is a monocular vision test comprising displaying lines and digits in ones of near, mid, and far peripheral areas represented by three circular zones marked on the display by the output of the display.

7. The method of claim 1, wherein
the selected at least one vision test mode comprises at least one of a glare test, a color distortion test, and a night vision test, and
the at least one visual display parameter comprises at least one of a background color, a line format, a line color, a line thickness, a peripheral display, an external lighting environment, and a display brightness.

8. The method of claim 7, wherein the glare test comprises, in a glare mode display of the display, controlling the output of the display by changing a background, a color gradient of lines in digits displayed relative to the background and in the central and peripheral regions of interest, and adapting visual display parameters comprising a glare effect, a brightness, a seven segment line display format, line colors that are a gradient of background colors and are predetermined to be visible to a normal vision, thickness weights of the lines, a size of a peripheral display to be larger moreso than that of a central display, the external lighting environment, and ranges of display brightness.

9. The method of claim 7, wherein the color distortion test comprises, in a color mode display of the display, changing a background, a color gradient of lines in digits displayed relative to the background and in the central and peripheral regions of interest, and adapting visual display parameters without glare and sharpness effect, a seven segment line display format, line colors that are a gradient of background colors and are predetermined to be visible to a normal vision, thickness weights of the lines, a size of a peripheral display to be larger than that of a central display, an average external lighting condition, and ranges of display brightness.

10. The method of claim 7, wherein the night vision test comprises, in a low light mode display of the display, changing a background, a color gradient of lines in digits displayed relative to the background and in the central and peripheral regions of interest, and adapting visual display parameters without glare and sharpness effect, a seven segment line display format, line colors that are a gradient of background colors and are predetermined to be visible to a normal vision, thickness weights of the lines, a size of a peripheral display to be larger than that of a central display, an average external lighting condition, and ranges of display brightness.

11. The method of claim 1, wherein
determining the type of cataract is further based on obtaining scores, including the score, of the central and peripheral regions of interest based on a plurality of weighing factors, a closeness of a response of the user vision relative to displayed lines and digits on the display, and a confidence level, and
the type of cataract indicates one of a posterior subcapsular cataract (C1), a cortical cataract (C2), a nuclear sclerotic cataract (C3), a combination cataract (C1+C2, C2+C3, and/or C1+C3), and a normal vision.

12. The method of claim 1, wherein performing the primary test comprises:
determining the type of cataract based on performing the primary test; and
iterating the primary test based on determining a difference between the type of cataract as determined based on the primary test as compared to the type of cataract as determined based on implementing the even further monitoring of the user vision relative to the display and in the state in which the at least one visual display parameter of the output of the display is adapted based on the obtained score and the selected at least one vision test mode.

13. The method of claim 1, wherein the one or more recommendations comprise at least one recommendation indicating a cataract type, an estimated time for surgery, an estimated time to retest for monitoring progression of cataract, a post-surgery test for analyzing improvement in vision, nutrients and food intake, lifestyle and day-to-day activities, and display settings based on any of a gender and a profession of the user.

14. An electronic device for determining a type of cataract, the electronic device comprising:
memory storing instructions;
a display; and
at least one processor operably coupled to the memory and the display, wherein the instructions, when executed by the at least one processor, cause the electronic device to:
perform a vision focus calibration for a user;
determine central and peripheral regions of interest on the display of the electronic device based on a fixated vision of the user;
perform a primary test comprising:
obtaining a score based on scoring the central and peripheral regions of interest,
selecting, from a plurality of vision test modes and based on the obtained score, at least one vision test mode, and
adapting, based on the obtained score and the selected at least one vision test mode, at least one visual display parameter;
determine the type of cataract of the user and a severity of the determined type of cataract based on the selected at least on vision test mode and the adapted at least one visual display parameter; and
providing information about an indication of the determined type of cataract, information about the determined severity, and information about one or more recommendations to the user.

15. The electronic device of claim 14, wherein the instructions, when executed by the at least one processor, cause the electronic device implement the vision focus calibration based on tracking any of:
one or more eyes of the user, and
facial features of the user.

16. The electronic device of claim 14, wherein the instructions, when executed by the at least one processor, cause the electronic device to monitor whether an environmental condition among the user and the display remains unchanged during a process comprising at least one of:
determining the central and peripheral regions of interest,
performing the primary test, and
implementing the even further monitoring of the user vision.

17. The electronic device of claim 14, wherein the primary test comprises:
monitoring a first eye of the user relative to a first portion of the output of the display, and
monitoring a second eye of the user relative to a second portion of the output of the display, wherein the first portion and the second portion are respectively at ones of opposite sides of the display.

18. The electronic device of claim 14, the selected at least one vision test mode comprises controlling the output of the display to be output in one of a glare mode display of the display, a color mode display of the display, and a low light mode display of the display.

19. The electronic device of claim 14, wherein performing the primary test comprises determining that the primary test and the even further monitoring in the selected at least on vision test mode result in different determinations of the type of cataract, and, based thereon, improving an accuracy of determining the type of cataract by reiterating the primary test and re-determining the type of cataract based on reiterating the primary test.

20. A non-transitory computer readable storage medium storing instructions which, when executed by at least one processor of an electronic device, cause the electronic device to:
perform a vision focus calibration for a user;
determine central and peripheral regions of interest on a display of the electronic device based on a fixated vision of the user;
perform a primary test comprising:
obtaining a score based on scoring the central and peripheral regions of interest,
selecting, from a plurality of vision test modes and based on the obtained score, at least one vision test mode, and
adapting, based on the obtained score and the selected at least one vision test mode, at least one visual display parameter;
determine the type of cataract of the user and a severity of the determined type of cataract based on the selected at least on vision test mode and the adapted at least one visual display parameter; and
providing information about an indication of the determined type of cataract, information about the determined severity, and information about one or more recommendations to the user.
